(19) United States
(12) Patent Application Publication
(10) Pub. No.: US 2024/0172967 A1
(43) Pub. Date: May 30, 2024

(54) SYSTEMS AND METHODS FOR PET MOBILITY DETECTION

(71) Applicant: Mars, Incorporated, McLean, VA (US)

(72) Inventors: Aletha CARSON, Gaston, OR (US); Lyndon Thomas Mcintosh REY, Calgary (CA); Christopher Andrew PROVAN, Chapel Hill, NC (US); Kathryn KRESNYE, Independence, MO (US); Lukmaan Abbas BAWAZER, Oakland, CA (US); Scott LYLE, San Francisco, CA (US)

(21) Appl. No.: 18/516,074

(22) Filed: Nov. 21, 2023

Related U.S. Application Data
(60) Provisional application No. 63/385,442, filed on Nov. 30, 2022.
(60) Provisional application No. 63/509,864, filed on Jun. 23, 2023.

(51) Int. Cl. A61B 5/11; G16H 50/20

(52) U.S. Cl. CPC A61B 5/1124; G16H 50/20

(57) ABSTRACT

A computer-implemented method for canine mobility detection is disclosed. The method includes processing mobility data captured by a device attached to a canine, analyzing canine data corresponding to the canine to determine at least one baseline canine, wherein the at least one baseline canine is similar to the canine, for each of the one or more metrics, comparing the one or more metrics of the canine to one or more baseline metrics of the at least one baseline canine, determining one or more scores for each of the one or more metrics, the one or more scores based on a normal range of the one or more metrics from the one or more baseline metrics of the at least one baseline canine, and displaying at least one alert on one or more user interfaces of a user device.

### CROSS-REFERENCE TO RELATED APPLICATION(S)

This application claims the benefit of priority to U.S. Provisional Application No. 63/385,442, filed on Nov. 30, 2022, and to U.S. Provisional Application No. 63/509,864, filed on Jun. 23, 2023, the entireties of which are incorporated herein by reference.

### TECHNICAL FIELD

Various embodiments of this disclosure relate generally to systems and methods for pet mobility detection.

### BACKGROUND

A pet owner may have difficulty determining whether a pet has mobility issues, such as arthritis. For example, the pet owner may struggle with distinguishing between the pet having mobility issues and the pet simply aging. Additionally, the pet owner may struggle with determining whether the pet has mobility issues that are normal for that particular breed, size, and the like. Current solutions do not allow for real-time analysis of the pet's mobility, and may not determine whether the pet's mobility issues are normal for similar pets with similar characteristics.

This disclosure is directed to addressing above-referenced challenges. The background description provided herein is for the purpose of generally presenting the context of the disclosure. Unless otherwise indicated herein, the materials described in this section are not admitted to be prior art, or suggestions of the prior art, by inclusion in this section.

### SUMMARY OF THE DISCLOSURE

According to certain aspects of the disclosure, methods and systems are disclosed for canine mobility detection.

In one aspect, an exemplary embodiment of a computer-implemented method for canine mobility detection is disclosed. The method may include processing, by one or more processors, mobility data captured by a device attached to a canine, the processing including determining one or more metrics based on the mobility data, the one or more metrics including velocity information, acceleration information, cadence information, and entropy information of the canine. The method may further include analyzing, by the one or more processors, canine data corresponding to the canine to determine at least one baseline canine, wherein the at least one baseline canine is similar to the canine. The method may further include, for each of the one or more metrics, comparing, by the one or more processors, the one or more metrics of the canine to one or more baseline metrics of the at least one baseline canine. The method may further include determining, by the one or more processors, one or more scores for each of the one or more metrics, the one or more scores based on a normal range of the one or more metrics from the one or more baseline metrics of the at least one baseline canine. The method may further include displaying, by the one or more processors, at least one alert on one or more user interfaces of a user device, the at least one alert indicating that an average of the one or more scores is above a threshold.

In a further aspect, an exemplary embodiment of a computer system for canine mobility detection is disclosed, the computer system comprising at least one memory storing instructions, and at least one processor configured to execute the instructions to perform operations. The operations may include processing mobility data captured by a device attached to a canine, the processing including determining one or more metrics based on the mobility data, the one or more metrics including velocity information, acceleration information, cadence information, and entropy information of the canine. The operations may further include analyzing canine data corresponding to the canine to determine at least one baseline canine, wherein the at least one baseline canine is similar to the canine. The operations may further include, for each of the one or more metrics, comparing the one or more metrics of the canine to one or more baseline metrics of the at least one baseline canine. The operations may further include determining one or more scores for each of the one or more metrics, the one or more scores based on a normal range of the one or more metrics from the one or more baseline metrics of the at least one baseline canine. The operations may further include displaying at least one alert on one or more user interfaces of a user device, the at least one alert indicating that an average of the one or more scores is above a threshold.

In a further aspect, an exemplary embodiment of a non-transitory computer-readable medium storing instructions that, when executed by at least one processor, cause the at least one processor to perform operations for canine mobility detection is disclosed. The operations may include processing mobility data captured by a device attached to a canine, the processing including determining one or more metrics based on the mobility data, the one or more metrics including velocity information, acceleration information, cadence information, and entropy information of the canine. The operations may further include analyzing canine data corresponding to the canine to determine at least one baseline canine, wherein the at least one baseline canine is similar to the canine. The operations may further include, for each of the one or more metrics, comparing the one or more metrics of the canine to one or more baseline metrics of the at least one baseline canine. The operations may further include determining one or more scores for each of the one or more metrics, the one or more scores based on a normal range of the one or more metrics from the one or more baseline metrics of the at least one baseline canine. The operations may further include displaying at least one alert on one or more user interfaces of a user device, the at least one alert indicating that an average of the one or more scores is above a threshold.

It is to be understood that both the foregoing general description and the following detailed description are exemplary and explanatory only and are not restrictive of the disclosed embodiments.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary platform environment, according to one or more embodiments.

FIG. 2 depicts an exemplary environment of a pet owner profile and corresponding pet profiles, according to one or more embodiments.

FIG. 3 depicts a flowchart of an exemplary embodiment for canine mobility detection, according to one or more embodiments.

FIG. 4 depicts an exemplary user interface 400 that displays a visual comparison between a pet's metrics and baseline metrics, according to one or more embodiments.

FIG. 5 depicts an exemplary diagram 500 of the chronic workflow. In some embodiments, a user interface may include one or more graphical representations, according to one or more embodiments.

FIG. 6 depicts an exemplary environment that may be utilized with techniques presented herein, according to one or more embodiments.

FIG. 7 depicts an example of a computing device that may execute the techniques described herein, according to one or more embodiments.

### DETAILED DESCRIPTION OF EMBODIMENTS

According to certain aspects of the disclosure, methods and systems are disclosed for canine mobility detection. Conventional techniques may not be suitable because conventional techniques may not allow for a real-time analysis and collection of the pet's mobility data. Moreover, conventional techniques may not allow for the comparison of the pet's mobility data with the mobility data of similar pets.

A need exists for an integrated hardware, software, and diagnostic solution for canine mobility detection. Such a solution may allow for the real-time tracking and analysis of canine mobility data, an efficient transfer of the canine mobility data between internal and external systems, and the ability for an analysis of the canine's veterinary data (e.g., medication data) in conjunction with the canine mobility data.

As will be discussed in more detail below, in various embodiments, systems and methods are described for canine mobility detection. The systems and methods may process mobility data captured by a device attached to a canine, the processing including determining one or more metrics based on the mobility data, the one or more metrics including velocity information, cadence information, acceleration information, and entropy information of the canine. The systems and methods may then analyze canine data corresponding to the canine to determine at least one baseline canine, wherein the at least one baseline canine is similar to the canine. The systems and methods may then include, for each of the one or more metrics, comparing the one or more metrics of the canine to one or more baseline metrics of the at least one baseline canine. The systems and methods may then determine one or more scores for each of the one or more metrics, the one or more scores based on a normal range of the one or more metrics from the one or more baseline metrics of the at least one baseline canine. The systems and methods may then display at least one alert on one or more user interfaces of a user device, the at least one alert indicating that an average of the one or more scores is above a threshold.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, a term such as “user” or the like generally encompasses a future pet owner, future pet owners, pet owner, and/or pet owners. A term such as “pet” or the like generally encompasses a domestic animal, such as a domestic canine, feline, rabbit, ferret, horse, cow, or the like. In exemplary embodiments, “pet” may refer to a canine.

### Exemplary Platform Environment

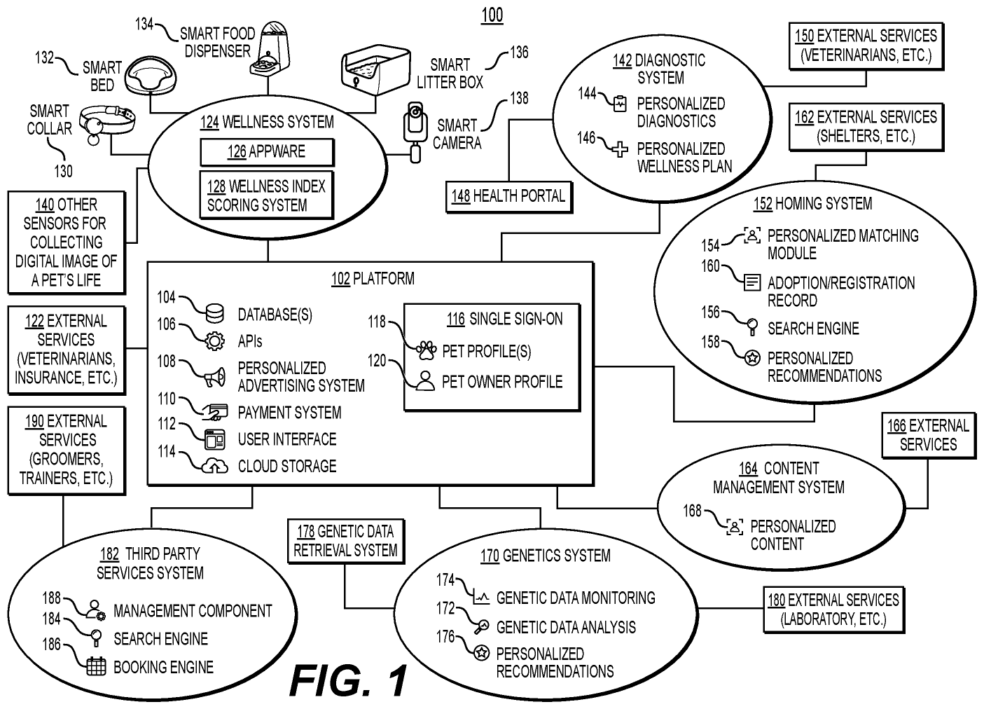

FIG. 1 depicts an exemplary platform environment 100 that may be utilized with the techniques presented herein. More specifically, environment 100 may provide an integrated hardware and software platform for improving pet digitalization by centralizing the pet's information.

Platform 102 may communicate with one or more external systems that may collect, manage, and store different types of pet data and/or pet owner data. Platform 102 may retrieve the pet data and/or pet owner data from the one or more external systems via APIs 106. In some embodiments, platform 102 may store the pet data and/or the pet owner data. For example, platform 102 may store the pet data in pet profile(s) 118. Additionally, for example, platform 102 may store the pet owner data in a pet owner profile 120. The one or more external systems may include at least one of a wellness system 124, a diagnostic system 142, a homing system 152, a content management system 164, a genetics system 170, and/or a third party services system 182. Such external systems are described in more detail below.

Platform 102 may also communicate with one or more external services. In some embodiments, platform 102 may communicate with the one or more external services via APIs 106. External services 122 may include, for example, one or more third party and/or auxiliary systems that integrate and/or communicate with the platform 102 in performing various pet tasks. For example, the external services 122 may include at least one of: a veterinarian, a pet insurance agency, a pet service provider, and the like.

Platform 102 may include database(s) 104 and/or cloud storage 114 that may store information corresponding to one or more pets and/or one or more pet owners. For example, the database(s) 104 and/or cloud storage 114 may store pet profile(s) 118 and/or pet owner profile 120. The database(s) 104 and/or the cloud storage 114 may be located internally or externally to platform 102.

Platform 102 may include a personalized advertising system 108 and/or a payment system 110. The personalized advertising system 108 may create and/or display personalized advertisements to the user. For example, the personalized advertisements may be created based on information contained in pet profile(s) 118 and/or pet owner profile 120. In some embodiments, the personalized advertising system 108 may display the personalized advertisements on a user interface 112 of the platform 102. The payment system 110 may allow the user to create a financial account for a pet and/or perform financial transactions for pet services and/or pet goods (e.g., using pet owner digital wallet 216).

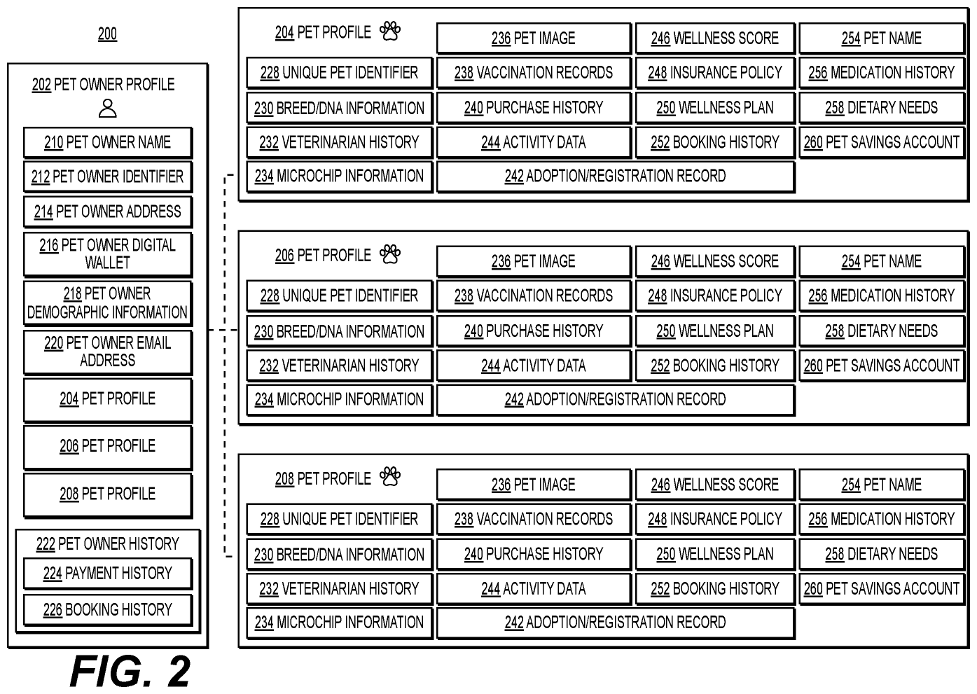

Platform 102 may include a single sign-on 116. The single sign-on 116 may include a unique identifier that may correspond to the pet profile(s) 118 and/or the pet owner profile 120. Each of the pet profile(s) 118 may include information corresponding to a particular pet. The pet owner profile 120 may include information corresponding to a particular pet owner. Additionally, the pet owner profile 120 and/or the pet profile(s) 118 may each have a corresponding avatar and/or virtual presence. The avatar and/or virtual presence may include different attributes that are shared by the pet owner and/or pets. The pet profile(s) 118 and pet owner profile 120 are described in further detail in the description of FIG. 2.

### Wellness System

The wellness system 124 may collect, manage, and/or display wellness data of a pet. The wellness system 124 may be an internal component or an external component of platform 102, where the wellness system 124 may communicate with platform 102 via APIs 106.

The wellness system 124 may collect data (e.g., mobility data) from one or more smart devices. The wellness system 124 may communicate with the one or more smart devices via one or more APIs. Additionally, in some embodiments, the wellness system may use appware 126 to facilitate the communication and/or the management of the one or more smart devices. For example, appware 126 may communicate with one or more smart devices that may run on an external system. Additionally, for example, appware 126 may run on a user device, where the appware 126 provides a user interface to display the data collected by the one or more smart devices. In some embodiments, appware 126 may manage one or more smart devices. The wellness system 124 may communicate with the one or more smart devices by sending one or more requests to the one or more smart devices. The requests may ask the one or more smart devices to send collected wellness data to the wellness system 124. In some embodiments, the one or more smart devices may automatically send wellness data (e.g., mobility data) to the wellness system 124. For example, the one or more smart devices may send the wellness data to the wellness system 124 at regular time intervals (e.g., every 30 seconds, every hour, every day, and the like) and/or whenever new wellness data is collected. In some embodiments, the wellness system 124 may store the wellness data in an internal or external storage. For example, the wellness system 124 may store the wellness data in databases 104 and/or cloud storage 114. Additionally, or alternatively, for example, the wellness system 124 may store the wellness data in the pet profile(s) 118 and/or the pet owner profile 120.

Upon receiving the wellness data from the one or more smart devices, a wellness index scoring system 128 may analyze the wellness data to determine a wellness score. The wellness index scoring system 128 may update the wellness score, where the updating is based on the most recently received wellness data. In some embodiments, the wellness index scoring system 128 may store the wellness score in one or more databases (e.g., database(s) 104) and/or cloud storage (e.g., cloud storage 114). For example, the wellness score may be stored in the pet profile(s) 118 and/or the pet owner profile 120. Additionally, or alternatively, the wellness system 124 may display the wellness score to the user. For example, the wellness system 124 may display the wellness score on a user interface of a user device. This may be accomplished by utilizing the appware 126. Additionally, or alternatively, the wellness system 124 may display the wellness score on one or more of the smart devices.

Example smart devices may include at least one of: a smart collar 130, a smart bed 132, a smart feeder 134, a smart litter box 136, a smart camera 138, and/or the other sensors for collecting a digital image of a pet's life 140.

The smart collar 130 may include a device and/or a sensor that may attach to a pet. For example, the smart collar 130 may attach around the pet's neck. The smart collar 130 may detect a pet's activity, location, and eating information, such as physical activity, location, eating habits, drinking habits, and the like. The smart collar 130 may detect and collect the pet's mobility information. The pet's mobility information may be processed to determine one or more metrics, such as the velocity, cadence, and entropy of the pet's gait. For example, the smart collar 130 may detect and collect the speed and direction at which the pet moves at any given point in time. Additionally, for example, the smart collar 130 may detect and collect data related to the inconsistency of the pet's gait (e.g., entropy of the pet's gait). For example, a higher entropy may imply a higher inconsistency detected in the pet's gait, which may be a sign of worse mobility. Conversely, a lower entropy measurement may imply a more consistent gait. The smart collar 130 may collect the activity, location, and eating information of the pet and send such information to the wellness system 124. In some embodiments, the smart collar 130 may automatically send the activity, location, and eating information to the wellness system 124 after a set period of time. In some embodiments, the smart collar 130 may send the activity, location, and eating information in response to a request from the wellness system 124.

The smart bed 132 may include a device and/or a sensor that may be included in a pet bed. The smart bed 132 may track sleeping information corresponding to the pet. The sleeping information may include the amount of time a pet sleeps in the smart bed 132, how frequently the pet gets up from the smart bed 132, if the pet tosses and turns while sleeping, and the like. The smart bed 132 may send such information to the wellness system 124. In some embodiments, the smart bed 132 may automatically send the sleeping information to the wellness system 124 after a set period of time. In some embodiments, the smart bed 132 may send the sleeping information in response to a request from the wellness system 124.

The smart feeder 134 may include a device and/or a sensor that may be included in a pet food feeder. The smart feeder 134 may track how much food is dispensed for the pet to eat. The smart feeder 134 may send such food dispensing information to the wellness system 124. In some embodiments, the smart feeder 134 may automatically send the food dispensing information to the wellness system 124 after a set period of time. In some embodiments, the smart feeder 134 may send the food dispensing information in response to a request from the wellness system 124.

The smart litter box 136 may include a device and/or a sensor that may be included in a litter box. The smart litter box 136 may track a pet's litter box information. The litter box information may include at least one of: how frequently the pet uses the smart litter box 136, what the pet does in the smart litter box 136, and the like. In some embodiments, the smart litter box 136 may automatically send the litter box information to the wellness system 124. In some embodiments, the smart litter box 136 may automatically send the litter box information to the wellness system 124 after a set period of time. In some embodiments, the smart litter box 136 may send the litter box information in response to a request from the wellness system 124.

The smart camera 138 may include a device and/or a sensor that may be included in a camera. The smart camera 138 may capture behavior information of a pet. The pet's behavior information may include physical activity, eating food from the pet's food dish, eating food from a source different from the pet's food dish, drinking from the pet's drinking dish, drinking from a source different from the pet's drinking dish, and the like. In some embodiments, the smart camera 138 may automatically send the behavior information to the wellness system 124 after a set period of time. In some embodiments, the smart camera 138 may send the behavior information in response to a request from the wellness system 124.

The other sensors for collecting a digital image of a pet's life 140 may include one or more devices and/or one or more sensors that collect data for a digital image of the pet's life. Example collected data may include information regarding the pet's eating behavior, sleeping behavior, drinking behavior, playing behavior, and the like. In some embodiments, the other sensors 140 may automatically send the collected data to the wellness system 124 after a set period of time. In some embodiments, the other sensors 140 may send the collected data in response to a request from the wellness system 124.

### Diagnostic System

The diagnostic system 142 may manage a pet's health information and provide personalized diagnostics 144 and/or a personalized wellness plan 146 to the user. The diagnostic system 142 may be an internal component or an external component of platform 102, where the diagnostic system 142 may communicate with platform 102 via APIs 106.

The diagnostic system 142 may manage a pet's heath information (e.g., vaccination records, medical records) by receiving the pet's health information from one or more external services 150 (e.g., veterinarians, clinics, pet hospital, pharmaceutical companies, and the like). The diagnostic system 142 may store the pet's health information in the pet profile(s) 118. In an embodiment, the diagnostic system 142 may communicate the pet's health information using APIs 106.

The diagnostic system may create personalized diagnostics 144 and/or a personalized wellness plan 146 based on the pet's health information. The personalized diagnostics 144 may include one or more diagnoses (e.g., ear infection, eye infection, and the like) of medical conditions for the pet. The personalized diagnostics 144 may be based on diagnoses made by the external services 150. In some embodiments, the personalized diagnostics 144 may be based on diagnoses made by one or more machine learning models. The personalized wellness plan 146 may include one or more recommendations regarding eating events, exercise events, health checks and wellness visits, and the like, which may be based on the pet's heath information. The personalized wellness plan 146 may be based on recommendations made by the external services 150. The personalized wellness plan 146 may be based on information included in the pet profile(s) 118. In some embodiments, the personalized wellness plan may be based on one or more recommendations made by one or more machine learning models.

The health portal 148 may provide access to one or more parties who wish to retrieve the personalized diagnostics 144, personalized wellness plan 146, and/or the pet's health information from the pet profile(s) 118. The health portal 148 may be internal or external to the diagnostic system 142. Additionally, the health portal 148 may include a user interface. For example, a groomer may access the health portal 148 to retrieve the pet's vaccination records from diagnostic system 142.

The diagnostic system 142 may communicate with external services 150, such as veterinarians, clinics, pet hospital, pharmaceutical companies, and the like. For example, an external service 150 (e.g., veterinarian) may send updated vaccine or medical records to the diagnostic system 142, where the diagnostic system 142 may then store such updated vaccine or medical records in the pet profile(s) 118. Additionally, for example, the diagnostic system may update the personalized diagnostics 144 and/or the personalized wellness plan 146 based on the updated vaccine or medical records.

In some embodiments, the diagnostic system 142 may receive and store the pet's vaccination and treatment information. For example, if a veterinarian administers a medication, vaccination, and/or alternative therapy to a pet, the external service(s) 150 may send the medical details to the diagnostic system 142, where the diagnostic system 142 may receive and store the medication, vaccination, and/or alternative therapy details (e.g., a medication dosage amount, a medication description, a medication administrator, and/or a medication administration timestamp). Additionally, in some embodiments, the diagnostic system 142 may store the medication details in the platform 102 (e.g., database(s) 104, cloud storage 114, and/or pet profile(s) 118). The diagnostic system 142 may also communicate the pet's vaccination and treatment information to the external service(s) 150. For example, the diagnostic system 142 may receive and store medication details from several external service(s) 142. The diagnostic system 142 may receive a request from one of the external service(s) 142 for the medication details of a particular pet. Upon receipt of the request, the diagnostic system 142 may communicate the medication details to the external service(s) 142.

In some embodiments, the diagnostic system 142 may include information to authenticate the pet. For example, social media websites frequently require that a user is authenticated in order to label the user as “verified” (e.g., a blue checkmark). The diagnostic system 142 may contain information corresponding to a physical examination of the pet. Such information may include authentication information of the pet. For example, the authentication information may include a confirmation of the pet's breed, gender, image, etc. Such authentication information may be used by a social media website to authenticate the pet as a “verified” user.

### Homing System

The homing system 152 may match a future pet owner with a pet and provide additional support for the future pet owner. The homing system 152 may be an internal component or an external component of platform 102, where the homing system 152 may communicate with platform 102 via APIs 106.

The homing system 152 may match a future pet owner with a particular pet using a personalized matching module 154 and/or a search engine 156. The personalized matching module 154 may use user information (e.g., user location, user age, and the like) from the future pet owner (e.g., from the pet owner profile 120) to automatically search for one or more pets that are best suited for the future pet owner. In some embodiments, the personalized matching module 154 may use one or more machine learning models to determine the best pet matches for the future pet owner. The search engine 156 may allow the future pet owner to search for one or more pets. The search engine 156 may include different search filters (e.g., filtering by breed, age, size, weight, and the like), which may allow the user to filter the results of the one or more pets.

Both the personalized matching module 154 and/or the search engine 156 may retrieve results from the external services 162. The external services 162 may include one or more of: a pet adoption agency, a shelter, a pet breeder, and the like. When the personalized matching module 154 and/or the search engine 156 is performing a search for one or more pets, the personalized matching module 154 and/or the search engine 156 may send one or more requests to the external services 162 for available pets that fit one or more parameters contained in the one or more requests. Upon receiving the one or more requests, the external services 162 may search one or more databases for one or more matching pets. The external services 162 may send a response to the personalized matching module 154 and/or the search engine 156. The response may include the one or more matching pets. Alternatively, for example, if no matching pets were found, the response may include an indicator that no matching pets were found. In some embodiments, the homing system 152 may store the one or more matching pets in a database, such as an internal database or an external database (e.g., database 104).

The homing system 152 may display the one or more matching pets to the future pet owner, along with an option for the future pet owner to adopt and/or purchase the one or more matching pets. The homing system 152 may also facilitate the adoption and/or purchase of the one or more matching pets. In some embodiments, the homing system 152 may communicate with the external services 162 to facilitate the adoption and/or purchase of the one or more matching pets.

Once the future pet owner purchases and/or adopts the pet, the homing system 152 may store and/or manage the pet's adoption/registration record 160. In some embodiments, the homing system 152 may receive all (or part of) the pet's adoption/registration record 160 from the external services. In some embodiments, the homing system 152 may store the pet's adoption/registration record 160 in the pet profile(s) 118. Additionally, or alternatively, the homing system 152 may store the pet's adoption/registration record in the pet owner profile 120. In some embodiments, the homing system 152 may store the pet's adoption/registration record 160 in an internal or external database.

The homing system 152 may provide additional support for the future pet owner by providing personalized recommendations 158 to the pet owner. The personalized recommendations 158 may be based characteristics of the pet that the future pet owner purchased and/or adopted. Example personalized recommendations 158 may include a recommended pet food, a recommended pet provider, recommended pet supplies, and the like. In some embodiments, the personalized recommendations 158 may be based on communications with one or more of the external services. For example, the homing system 152 may communicate with the content management system 164 to receive personalized content 168, and then make personalized recommendations 158 based on the personalized content 168.

### Content Management System

The content management system 164 may provide personalized content 168 to a user. The content management system 164 may be an internal component or an external component of platform 102, where the content management system 164 may communicate with platform 102 via APIs 106.

The content management system 164 may retrieve personalized content 168 and display such personalized content 168 to the user. The personalized content 168 may include at least one of: an article, a blog post, an online forum, an advertisement, and the like. The personalized content 168 may also include recommendations that are specific towards the pet and/or user. The recommendations may include food recommendations, activity recommendations, product recommendations, resource recommendations (e.g., books, articles, and the like), third party services recommendations (e.g., groomer, trainer, boarding), and the like. The personalized content 168 may be personalized based on pet profile(s) 118 and/or pet owner profile 120. The content management system 164 may display the personalized content 168 via a user interface of a user device. In some embodiments, the content management system 164 may retrieve the personalized content 168 from the external services 166. The external services 166 may include an electronic magazine, one or more databases, one or more social media posts, and the like. In some embodiments, the content management system 164 may retrieve the personalized content 168 from other sources, such as database(s) 104, cloud storage 114, and personalized advertising system 108. In some embodiments, the content management system 164 may create personalized content 168 based on communications with the other external systems (e.g., wellness system 124, diagnostic system 142, homing system 152, genetics system 170, third party services system 182, etc.). For example, the content management system 164 may receive the personalized wellness plan 146 from diagnostic system 142. The personalized content 168 may then be based on (or include) information from the personalized wellness plan 146.

### Genetics System

The genetics system 170 may analyze and/or monitor a pet's genetic data. The genetics system 170 may be an internal component or an external component of platform 102, where the genetics system 170 may communicate with platform 102 via APIs 106.

The genetics system 170 may include genetic data analysis 172, genetic data monitoring 174, and/or personalized recommendations 176. Additionally, the genetic data analysis 172 and/or the genetic data monitoring 174 may communicate with external services 180 to assist with the analysis and/or the monitoring of the genetic data. The external services may include a laboratory, a clinic, a veterinarian, and the like.

The genetic data analysis 172 may receive genetic data belonging to a pet. In some embodiments, the genetic data analysis 172 may receive the genetic data from a genetic data retrieval system 178. The genetic data retrieval system 178 may retrieve and store genetic data belonging to one or more pets. Additionally, the genetic data analysis may receive genetic data from the genetic data retrieval system 178, where the received genetic data is used in the analysis of the genetic data belonging to the pet. The genetic data analysis 172 may analyze the genetic data to determine abnormalities, potential genetic traits, familial relationships, and the like. In some embodiments, the genetic data analysis 172 may communicate with external services 180 to assist with the analysis of the genetic data. For example, the genetic data analysis 172 may send genetic data information to a laboratory for the laboratory to perform the analysis of the genetic data.

The genetic data monitoring 174 may monitor the genetic data belonging to a pet to determine any changes in the genetic data. For example the genetic data monitoring 174 may receive new genetic data and compare the new genetic data to previously stored genetic data. The comparing may lead the genetic data monitoring 174 to determine that there is an abnormality or an improvement in the genetic data. In some embodiments, the genetic data monitoring 174 may communicate with the external services 180, in order for the external services 180 to analyze the genetic data and determine if there are any changes.

The genetics system 170 may provide personalized recommendations 176 to the user. For example, the genetics system 170 may provide personalized recommendations 176 to the user via a user interface of a user device. In some embodiments, the personalized recommendations may be based on the genetic data analysis 172 and/or the genetic data monitoring 174. The personalized recommendations 176 may include a pet food recommendation, an exercise recommendation, a pet item recommendation, health checks or wellness visits, and the like. In some embodiments, the personalized recommendations 176 may be based on communications with one or more of the external services. For example, the genetics system 170 may communicate with the diagnostic system 142. The genetics system 170 may send a request to the diagnostic system 142 for a personalized wellness plan 146. The request may include, for example, the genetic data analysis 172 and/or the genetic data monitoring 174. The diagnostic system 142 may communicate a personalized wellness plan 146 to the genetics system 170, where the personalized wellness plan 146 may be based on the genetic data analysis 172 and/or the genetic data monitoring 174. The genetics system 170 may make personalized recommendations 176 to the user based on the personalized wellness plan 146.

In some embodiments, the genetics system 170 may include information to authenticate the pet. For example, social media websites frequently require that a user is authenticated in order to label the user as “verified” (e.g., a blue checkmark). The genetics system 170 may contain information corresponding to a physical examination of the pet. Such information may include authentication information of the pet. For example, the authentication information may include a confirmation of the pet's breed, gender, image, etc. Such authentication information may be used by a social media website to authenticate the pet as a “verified” user.

### Third Party Services System

The third party services system 182 may allow a user to search for and reserve different external services 190, such as groomers, trainers, veterinarians, holistic care (e.g., nutritionist, naturopathic), and the like. The third party services system 182 may be an internal component or an external component of platform 102, where the third party services system 182 may communicate with platform 102 via APIs 106.

The third party services system 182 may include a search engine 184, a booking engine 186, and/or a management component 188.

The search engine 184 may allow the user, such as a pet owner, to search for external services 190 to reserve for the user's pet. The search engine 184 may include filtering functionality to facilitate a fine-tuned search. The filtering functionality may include universal filtering and/or service specific filtering. For example, the universal filtering may include filtering the external services 190 by location, price range, and/or ratings. Additionally, for example, the service specific filtering may include filtering the external services 190 by breed specialty, health issues, and/or behavioral needs.

The booking engine 186 may allow the user to reserve the external services 190. For example, after using the search engine 184 to search for external services 190, the user may use the booking engine 186 to reserve a particular service of the external services 190. The booking engine 186 may present open dates and time slots, which may correspond to the selected external service 190. The user may then user the booking engine 186 to select a date and/or time from the displayed open dates and time slots. Upon the finalization of the booking, the user may receive an instant confirmation of the booking, such as via text or email. The user may also have the ability to instantly pay for the booked service. Alternatively, the user may be able to pay upon the finalization of the service. The user may be able to upload photos and include notes to the external service 190. For example, the user may upload dog photos to a groomer, or make a note that the user's dog has a limp.

The management component 188 may provide functionality to manage different external services 190. For example, the management component 188 may provide the functionality for external services 190 to register and/or be removed from the third party services system 182. The management component 188 may communicate with one or more databases (e.g., database(s) 104) and/or cloud storage (e.g., cloud storage 114) to store information (e.g., a name, a business identifier, a specialty, and the like) corresponding to the external services 190.

### Exemplary Pet Owner Profile and Pet Profile(s)

FIG. 2 depicts an exemplary environment 200 of a pet owner profile 202 and corresponding pet profiles that may be utilized with the techniques presented herein. Notably, exemplary platform environment 200 may complement exemplary platform environment 100, with pet owner profile 202 corresponding to pet owner profile 120 of FIG. 1. Additionally, pet profile 204, pet profile 206, and/or pet profile 208 may correspond to pet profiles(s) 118 of FIG. 1.

Pet owner profile 202 may include at least one of: a pet owner name 210, a pet owner identifier 212, a pet owner address 214, a pet owner digital wallet 216, pet owner demographic information 218, a pet owner email address 220, at least one pet profile (e.g., pet profile 204, pet profile 206, pet profile 208) and/or at least one identifier associated with the at least one pet profile, and/or a pet owner history 222. The pet owner name 210 may include a name of the pet owner. The pet owner identifier 212 may include a unique identifier that may be used to locate the pet owner profile 202. In some embodiments, the pet owner identifier 212 may allow for tracking of some or all of the user's activities. The pet owner address 214 may include a physical address of the pet owner. The pet owner digital wallet 216 may include payment information, such as credit card information, cryptocurrency information, and the like. The pet owner demographic information 218 may include a particular demographic of the pet owner. The pet owner email address 220 may include an email address of the pet owner. The pet owner profile may include at least one pet profile (e.g., pet profile 204, pet profile 206, pet profile 208). In some embodiments, in lieu of including an entirety of the at least one pet profile, the pet owner profile 202 may include at least one identifier associated with the at least one pet profile (e.g., unique pet identifier 228). Each of the pet profiles may correspond to a pet that belongs to the pet owner. The number of pet profiles may be dynamic, where the pet profiles may adjust according to the number of pets that belong to the user.

The pet owner history 222 may include a payment history 224 and/or a booking history 226. The payment history 224 may include financial transactions of the pet owner. In some embodiments, the payment history 224 may correspond to activity of the pet owner digital wallet 216. In some embodiments, the payment history 224 may be tracked and analyzed to provide for targeted advertising (e.g., of personalized advertising system 108) and/or recommendations to the pet owner. The booking history 226 may include previous bookings of third party services that were made by the user. In some embodiments, the booking history 226 may be tracked and analyzed to provide for targeted advertising (e.g., of personalized advertising system 108) and/or recommendations to the pet owner.

Pet profile 204, pet profile 206, and/or pet profile 208 may each correspond to a different pet that belongs to the pet owner of the pet owner profile 202. The pet owner may have more or less than three pets. The number of pet profiles may be dynamic, where the number of pet profiles corresponds to the number of pets that belong to the pet owner. In some embodiments, the pet owner may want only a subset of the pet owner's pets to have pet profiles.

Pet profiles 204, 206, and/or 208 may each include at least one of: a unique pet identifier 228, breed/DNA information 230, veterinarian history 232, microchip information 234, a pet image 236, vaccination records 238, a purchase history 240, an adoption/registration record 242, activity data 244, a wellness score 246, an insurance policy 248, a wellness plan 250, a booking history 252, a pet name 254, medication history 256, dietary needs 258, and/or a pet savings account 260.

The unique pet identifier 228 may include a unique identifier that may be used to locate the corresponding pet profile (e.g., pet profiles 204, 206, and/or 208). In some embodiments, the unique pet identifier 228 may allow for tracking of some or all of activities corresponding to the pet.

The pet image 236 may include to a photograph, drawing, virtual presence, and/or avatar of the pet. The pet name 254 may include the name of the pet and/or any nicknames. The insurance policy 248 may include a pet insurance policy for the pet. The purchase history 240 may include purchases made for the pet. The pet savings account 260 may include a financial savings account for the pet. In some embodiments, the pet image 236, the pet name 254, the purchase history 240, pet savings account 260, and/or the insurance policy 248 may have been received from one or more of the external systems.

The breed/DNA information 230 may correspond to the breed and/or DNA information of the pet. In some embodiments, the breed/DNA information 230 may have been received from one or more of the external systems. For example, the breed/DNA information 230 may have been received from genetics system 170.

The veterinarian history 232 may include the details of the pet's visit(s) to a veterinarian. The veterinarian history 232 may also include notes from the vet and/or possible diagnoses and treatments. The vaccination records 238 may include one or more vaccination records of vaccinations administered to the pet. The medication history 256 may include details of the medications that the pet currently takes and/or has taken in the past. The dietary needs 258 may include information regarding food that the pet should eat and/or food that the pet should avoid. The wellness plan 250 may correspond to a wellness plan for the pet. In some embodiments, the wellness plan 250 may have been determined based on personalized wellness plan 146. In some embodiments, the veterinarian history 232, vaccination records 238, dietary needs 258, wellness plan 250, and/or the medication history 256 may have been received from one or more of the external systems. For example, the veterinarian history 232, vaccination records 238, dietary needs 258, wellness plan 250, and/or the medication history 256 may have been received from diagnostic system 142.

The microchip information 234 may include a microchip number of the pet. For example, the microchip may have been inserted into the pet to track the pet. The adoption/registration record 242 may include documentation of the adoption or purchase of the pet. In some embodiments, the microchip information 234 and/or adoption/registration record 242 may have been received from one or more of the external systems. For example, the microchip information 234 and/or adoption/registration record 242 may have been received from homing system 152.

The activity data 244 may include data corresponding to mobility data, physical activities, sleep activities, and/or food activities of the pet. For example, the mobility data and/or activity data may be collected by a smart collar 130, a smart bed 132, a smart feeder 134, a smart litter box 136, a smart camera 138, and/or the other sensors for collecting a digital image of a pet's life 140. The wellness score 246 may include data corresponding to a wellness score produced by wellness index scoring system 128. In some embodiments, the activity data 244 and/or the wellness score 246 may have been received from one or more of the external systems. For example, the activity data 244 and/or the wellness score 246 may have been received from wellness system 124.

The booking history 252 may include data corresponding to one or more bookings of a third party service (e.g., groomer, trainer, and the like). In some embodiments, the booking history 252 may have been received from one or more of the external systems. For example, the booking history 252 may have been received from the third party services system 182.

### Exemplary Method for Canine Mobility Detection

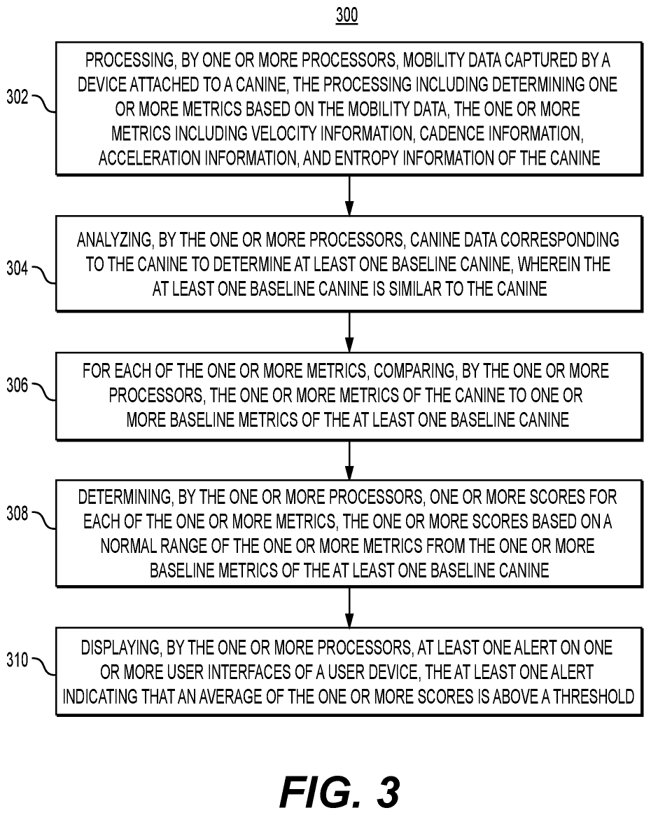

FIG. 3 illustrates an exemplary method 300 of an exemplary embodiment for canine mobility detection, according to one or more embodiments. Notably, method 300 may be performed by one or more processors of a server that is in communication with one or more user devices and other external system(s) via a network. However, it should be noted that method 300 may be performed by any one or more of the server, one or more user devices, or other external systems.

The method may include processing, by one or more processors, mobility data captured by a device attached to a canine, the processing including determining one or more metrics based on the mobility data, the one or more metrics including velocity information, cadence information, acceleration information, and entropy information of the canine (Step 302). The device may include one or more of: smart collar 130, smart bed 132, smart feeder 134, smart litter box 136, smart camera 138, and/or the other sensors for collecting a digital image of a pet's (e.g., canine's) life 140. For example, the device (e.g., an accelerometer) may be attached to a collar of the canine. Additionally, the device may include one or more sensors that track mobility data of the canine. Additionally, the mobility data captured by the device may be transmitted to a data store (e.g., a centralized repository). The mobility data may then be stored in the data store.

The mobility data may then be processed to determine one or more metrics based on the mobility data, where the one or more metrics may include velocity information, cadence information, acceleration information, and entropy information of the canine. The velocity information may include acceleration information. For example, the velocity information may correspond to how quickly and how fast a pet moves. The acceleration information may correspond to how a pet accelerates when moving. The acceleration information may include how quickly a pet accelerates, how much a pet accelerates, how frequently a pet accelerates, and the like. The entropy information may include the consistency of a pet's movements. The entropy information may include the size and/or breed of the pet. Additionally, for example, if the size of the pet is not known, the size of the pet may be predicted based on a Welch power spectrum analysis. In some embodiments, the size of the pet may include a corresponding size category, where one or more size categories may be determined based on a shifting scale. For example, the method may include determining one or more size categories based on continuous size scaling integrated into a model, where the model may determine a size category corresponding to the pet based on the size of the pet and the one or more size categories. The cadence information may correspond to the frequency at which a gait cycle of the pet occurs. The cadence information may indicate a derivation from a normal pet's movements. For example, an uneven cadence may likely indicate a limp. A lower cadence may indicate a pet that walks slower when compared with similar pets. The one or more metrics may include data of a particular pet action, such as running. The one or more metrics may further include stride time, stance time, step time, swing time, and/or step length of the canine.

In some embodiments, the entropy information may be calculated by segmenting, in non-overlapping frames, the mobility data into one or more windows. The means and entropies may then be calculated for each constituent signal. The magnitude of the three dimensional acceleration (and the corresponding entropy) may also be calculated for each window by determining the square root of the sum of squares of the three directional accelerometer axes. The overall values may be calculated from the means of each window.

In some embodiments, the method may include filtering, by a deep learning L1 activity prediction model, the mobility information to identify one or more suspected timeframes, where the one or more suspected timeframes correspond to canine mobility (e.g., walking). For example, canine walking data may be determined by analyzing the mobility data to determine one or more timeframes where L1 activity predictions may predict walking as the most likely activity for a timespan in question.

Additionally, or alternatively, in some embodiments, the method may include analyzing, by the one or more processors, the mobility data to determine at least one portion of the mobility data that includes walking data, where the walking data corresponds to a user taking a pet on a walk. For example, one or more sensors, which may be attached to the user and/or the pet, may detect that the user is taking the pet on a walk. The method may further include removing, by the one or more processors, the at least one portion from the mobility data.

Additionally, or alternatively, in some embodiments, the method may include analyzing, by the one or more processors, the mobility data to determine at least one portion of the mobility data that does not include walking data. For example, walking data may be determined by applying a windowed mean-of-maxes amplitude filter (e.g., remove any window where the mean of peak amplitudes post peak-fining is below a threshold), removing windows where most dominant harmonics are a higher frequency than expected, applying an area filter and removing windows that may have an absolute under-curve area below a certain threshold. The method may further include removing, by the one or more processors, the at least one portion from the mobility data.

The method may include analyzing, by the one or more processors, canine data corresponding to the canine to determine at least one baseline canine, wherein the at least one baseline canine is similar to the canine (Step 304). The canine data may include age data of the canine, breed data of the canine, weight data of the canine, one or more risk factors of the canine (e.g., risk factors for an arthritis diagnosis), or medical history of the canine (e.g., acute injury). In some embodiments, the analyzing may include utilizing a trained machine-learning model to determine the at least one baseline canine. For example, the canine data may be input into the trained machine-learning model. Upon receiving the input, the trained machine-learning model may analyze the canine data and determine at least one baseline canine that is similar to the canine data.

In some embodiments, the analyzing may include segmenting, by the one or more processors, the mobility data into a plurality of windows based on at least one time interval. For example, the mobility data may be segmented into six second chunks at a five second overlap. The analyzing may further include analyzing, by the one or more processors, each of the plurality of windows to determine whether a threshold number of the plurality of windows is below a window threshold. For example, the analyzing may include determining if 25%+ of the windows are below a threshold (e.g., around 0.05 g). The analyzing may further include removing, by the one or more processors, each of the plurality of windows that falls below the threshold.

In some embodiments, the analyzing may further include determining, by the one or more processors, a standard deviation for each of the plurality of windows. The analyzing may further include removing, by the one or more processors, each of the plurality of windows from the mobility data that falls below a standard deviation threshold. For example, windows that have a standard deviation that is less than 0.1 g may be removed from the mobility data.

In some embodiments, the analyzing may further include determining, by the one or more processors, a highest oscillation frequency of the plurality of windows. For example, the highest oscillation frequency may be determined using Welch's power spectra density algorithm (PSD). In some embodiments, the analyzing may further include analyzing, by the one or more processors, the highest oscillation frequency to determine whether the highest oscillation frequency falls outside of an oscillation range. For example, the range may include 1 Hz-5 Hz, where anything less or more may be unrealistic. In some embodiments, the analyzing may further include, in response to determining that the highest oscillation frequency does fall outside of the oscillation range, removing, by the one or more processors, the window of the plurality of windows that corresponds to the highest oscillation frequency from the mobility data. For example, if the highest oscillation frequency is outside of the range (e.g., 1 Hz-5 Hz), the window(s) corresponding to the highest oscillation frequency may be removed from the mobility data.

In some embodiments, the method may include classifying, by the one or more processors, based on analyzing the canine data, the canine as a small canine, a medium canine, a large canine, or a giant canine. For example, a power frequency extraction methodology may automatically classify the canine as small, medium, large, or giant. In some embodiments, a trained machine-learning model may determine the classification of the canine. For example, the trained machine-learning model may receive some or all of the canine data. Upon receiving some or all of the canine data, the trained machine-learning model may determine a classification of the canine based on baseline canine data of similar canines.

The method may include, for each of the one or more metrics, comparing, by the one or more processors, the one or more metrics of the canine to one or more baseline metrics of the at least one baseline canine (Step 306). The one or more baseline metrics may include velocity information, cadence information, acceleration information, and entropy information of the at least one baseline canine. The one or more baseline metrics may further include mobility data of a particular pet action, such as running. The one or more baseline metrics may further include stride time, stance time, step time, swing time, and/or step length of the canine. Additionally, any of the embodiments described in Appendices A-C may be utilized for this or any of the limitations described herein.

The method may include determining, by the one or more processors, one or more scores for each of the one or more metrics, the one or more scores based on a normal range of the one or more metrics from the one or more baseline metrics of the at least one baseline canine (Step 308). For example, each of the one or more metrics may have a corresponding score. The score may be based on an average number of standard deviations the metric is from the baseline metric. The normal range may indicate normal mobility for the canine. The normal range may include an upper mobility bound and a lower mobility bound. For example, the normal range may include +/−standard deviation from the average mobility of baseline canines. In some embodiments, the scores that fall outside of the normal range may be considered to have invalid results. Additionally, as described above, any of the embodiments described in Appendices A-C may be utilized for this or any of the limitations described herein.

The method may include displaying, by the one or more processors, at least one alert on one or more user interfaces of a user device, the at least one alert indicating that an average of the one or more scores is above a threshold (Step 310). The threshold may have been predetermined and/or may be different depending on the canine data. The alert may indicate that the canine may be experiencing symptoms of osteoarthritis or other mobility diseases. The alert may indicate that the user should notify the canine's veterinarian of a possible mobility issue. Additionally, the alert may be sent to one or more external services (e.g., wellness system 124, a diagnostic system 142, a homing system 152, a content management system 164, a genetics system 170, and/or a third party services system 182.).

In some embodiments, the method may further include receiving, by the one or more processors, canine veterinary data associated with the canine from one or more external systems. The canine veterinary data may correspond to one or more of the canine's veterinary records (e.g., treatment data). The canine veterinary data may include canine medication data that may correspond to a medication, vaccination, and/or alternative treatment that was administered to the canine. The canine medication data may include at least one medication dosage amount, at least one medication description, at least one medication administrator, or at least one medication administration timestamp. The method may further include receiving, by the one or more processors, baseline canine veterinary data associated with the at least one baseline canine from one or more data stores. The method may further include analyzing, by the one or more processors, the canine veterinary data, the one or more scores, the baseline canine veterinary data, and the one or more baseline metrics. The method may further include, based on the analyzing, determining, by the one or more processors, that the canine has a mobility issue (e.g., osteoarthritis). For example, the analyzing may determine whether the canine's mobility data in view of the veterinary data is normal or abnormal when compared to a similar canine who has similar veterinary data. The method may further include displaying, by the one or more processors, at least one mobility alert indicating the mobility issue on the one or more user interfaces of the user device. Additionally, for example, the mobility alert may indicate the impact of the medication on the canine's mobility. For example, the mobility alert may indicate whether the medication is helping or not helping the canine. Additionally, for example, the mobility alert may indicate whether the canine is compliant with the medication and/or alternative therapies.

An exemplary embodiment may include utilizing a mobility detection algorithm in combination with markerless kinematic video to determine a mobility recommendation. Specifically, the embodiment may include receiving and analyzing pet mobility data to determine whether the pet may have a possible mobility issue. For example, the pet mobility data may include a markerless kinematic video that was taken by a user (e.g., a pet parent) with the user's cell phone. Additionally, or alternatively, the pet mobility data may have been captured by one or more sensors. The embodiment may further include analyzing the mobility data to determine a potential mobility issue and, based on the analyzing, determine recommended care. The recommended care may include a care plan that includes follow-up for continuous monitoring with additional data collected (e.g., additional parameters of mobility data). Based on an initial analysis of the pet mobility data, a display may prompt a user (e.g., a pet parent) to take a video of the pet's movements. In response to receiving the prompt, the user may capture a markerless kinematic video of the pet's movements. The user may capture the markerless kinematic video using a cell phone, laptop, camera, and the like. A mobility algorithm then analyze the markerless kinematic video to determine a mobility result. The mobility result may include: (1) a mobility issue has been verified, (2) a mobility issue is inconclusive, or (3) the mobility appears normal. If the mobility issue has been verified, the system may display a corresponding recommendation for the user to seek veterinary care for the pet. If the mobility issue is inconclusive, the system may display a corresponding recommendation for the user to repeat the markerless kinematic video and/or for the user to schedule a recheck of the markerless kinematic video. If the mobility appears normal, the mobility algorithm may be tuned.

Although FIG. 3 shows example blocks of exemplary method 300, in some implementations, the exemplary method 300 may include additional blocks, fewer blocks, different blocks, or differently arranged blocks than those depicted in FIG. 3. Additionally, or alternatively, two or more of the blocks of the exemplary method 300 may be performed in parallel.

### Exemplary User Interface

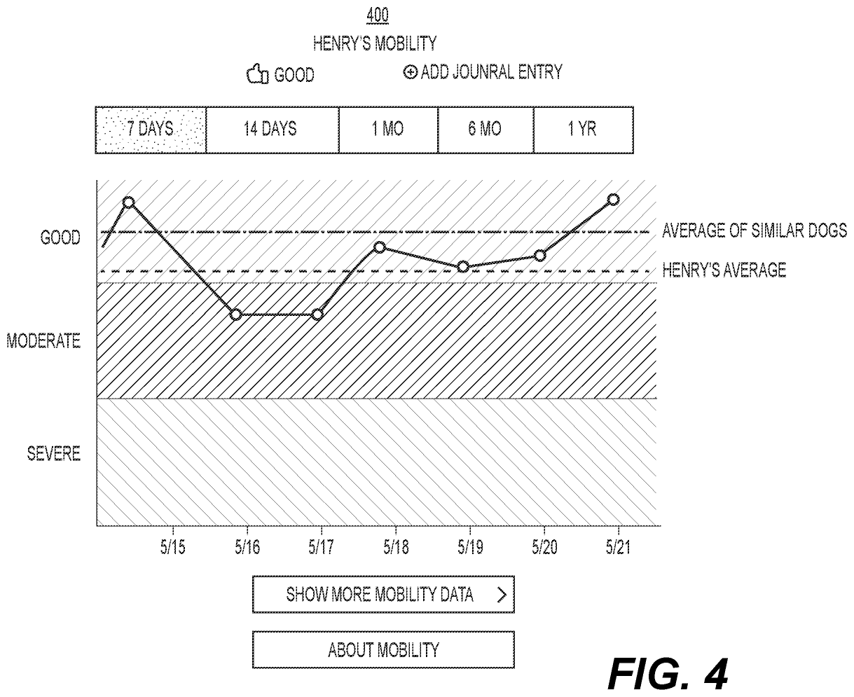

FIG. 4 depicts an exemplary user interface 400 that displays a visual comparison between a pet's metrics and baseline metrics, according to one or more embodiments. The user interface 400 may increase a pet owner's compliance with a pet's care plan by presenting a visualization of the pet's response to the care recommendations. The pet owner may see via the user interface 400 that the pet is responding to the care recommendations, and as a result, the pet owner may be incentivized to continue to follow the care recommendations. Additionally, the user interface 400 may provide information that may further a veterinarian's diagnostics of the pet.

The exemplary user interface 400 may include an indicator of how the pet's mobility compares to other pets. For example, “Good” may be displayed if a pet's average mobility is similar to other pets, whereas “Moderate” may be displayed if a pet's average mobility is below other pets, and “Severe” may be displayed if a pet's average mobility is significantly below other pets.

The user interface 400 may include at least one graphical representation that may include at least one mapping (e.g., data points and/or a line) that displays a comparison of the pet's average mobility to other similar pets' mobility. The graphical representation may include at least one mapping (e.g., data points and/or line) that displays a pet's average mobility. The graphical representation may include at least one mapping (e.g., data points and/or line) that displays an average mobility of similar pets. For example, one or more pets may be determined to be similar based on the pet's size, breed, and the like. In some embodiments, the user interface 400 may include an option (e.g., a toggle) to display the average mobility of similar pets.

The graphical representation may include a y-axis that indicates a mobility score range. For example, the y-axis may indicate the mobility score numerically and/or by a label (e.g., “good,” “moderate,” and/or “severe”). Additionally, an upper bound and/or a lower bound of mobility metrics may be set, where a pet that has a metric that falls outside of the upper bound or the lower bound may be determined to have invalid results (instead of extremely bad mobility). In some embodiments, if the model predicts a mobility score that is out of range and/or a complete outlier (e.g., a value that is not comparable for any pet), the user interface 400 may show nothing or display a null state. The graphical representation may include an x-axis that indicates one or more time periods (e.g., hours, days, and the like). The user interface 400 may present one or more time periods as options for the user. For example, the user may be presented the option to display mobility data for the past 7 days, 14 days, 1 month, 6 months, and/or 1 year.

The user interface 400 may include an option to display additional mobility data, where the additional mobility data may include data corresponding to the cadence or consistency of the pet. The user interface 400 may also include an “About Mobility” option, which when selected, may provide additional information explaining the meaning of the pet's mobility score, and/or provide additional information regarding the mobility of the pet and common issues the pet may experience.

Additionally, or alternatively, the user interface 400 may include one or more survey instruments that may capture additional data about the pet's mobility. For example, the survey instruments may include one or more prompted questions. A user may be able to input a response to the prompted questions via user interface 400. The prompted questions may be correlated and/or linked to content created by external or internal systems (e.g., content management system 164). For example, the prompted questions may be linked to short blogs about understanding acute and chronic mobility issues. In some embodiments, the linking may include smart logic that links the content.

The user interface 400 may include the functionality to allow a user to provide one or more journal entries relating to the pet (e.g., “add journal entry”). For example, the journaling capability may include displaying one or more questions that may have a time-bound start/stop time of when an event occurred and/or when a change was first noticed. In some embodiments, the responses to such questions may be converted into one or more timeline tags on the user interface 400 (e.g., a mobility tracker).

Additionally, or alternatively, the user interface 400 may provide one or more alerts. The alerts may include positive alerts and/or alerts expressing concern. The positive alerts may express positive reinforcement to the user and/or pet. The alerts expressing concern may indicate that there may be a particular issue with the pet. For example, one or more smart alerts (e.g., celebrating a dog's movement getting closer to normal) may be displayed via user interface 400. The positive reinforcement may result in improving the compliance of the pet owner following the pet's care recommendation. Other alerts may express concern when the pet's mobility drops below a threshold.

Additionally, or alternatively, the user interface 400 may implement a chronic workflow. The chronic workflow may include a rule that a baseline may need to be established before a chronic workflow is available for a pet. For example, a rule may indicate that 14 days of mobility data may need to be captured prior to making the chronic workflow available for the pet. The chronic workflow may be triggered when a pet has a lower mobility average than similar pets. Additionally, or alternatively, the chronic workflow may be triggered when a decline in mobility has been occurring over a period of time (e.g., 8 weeks) when the pet previously had a normal baseline. The chronic workflow may include the following questions:
Does your dog limp or appear stiff after exercise?
Do you think your dog shows signs of pain?
Is your dog reluctant to climb stairs or jump?
Does your dog have difficulty in rising from a resting position?
Have you noticed a change in your dog's behavior?
Does your dog tire easily or lag behind during walks?
Has your dog ever been injured? If so, when? What was the injury? Do you know an approximate date? Did your pet make a full recovery?
Have you ever given your dog medication for pain? If so, when?
Has your dog gained weight in the last year?
Has a veterinarian ever diagnosed your pet with a joint or mobility problem?

If the user indicates “yes” to one or more of the above questions, the user interface 400 may display one or more links to chronic mobility educational content. Such content may include treatment options, such as at home treatment options to improve and/or maintain the pet's mobility. In some embodiments, if the system determines that the pet has gained weight and/or the pet's weight is above a threshold, the user interface 400 may display weight loss content and/or enter a weight loss/mobility care mode.

If the user indicates “no” to one or more of the above questions, the user interface 400 may prompt the user to provide more information. The user interface 400 may prompt the user to consent to the use of the user's data for additional training. The user interface 400 may prompt the user for at least one of the following: submit at least one picture of the pet (e.g., side view of the pet, level view of the pet, and/or top view of the pet), verify the weight of the pet, and/or any other pet issues (e.g., blind, neurological problems, seizures, and/or other health issues that could be misclassified as mobility issues).

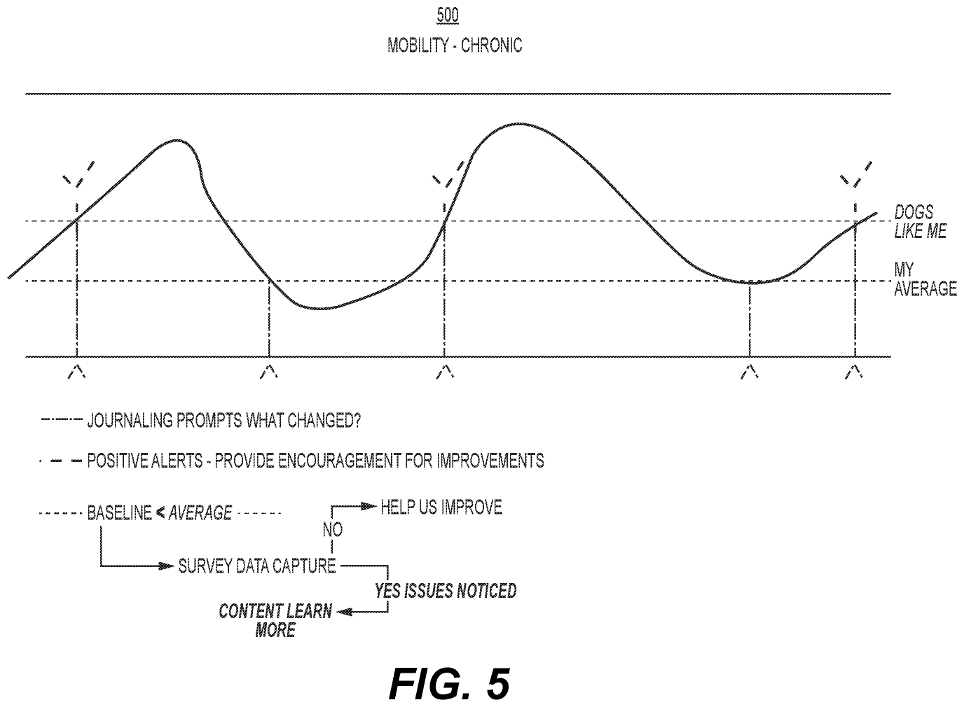

FIG. 5 depicts an exemplary diagram 500 of the chronic workflow. In some embodiments, a user interface may include one or more graphical representations, according to one or more embodiments. The user interface may include at least one graphical representation that may include at least one mapping (e.g., data points and/or a line) that displays a comparison of the pet's average mobility to other similar pets' mobility. The graphical representation may include at least one mapping (e.g., data points and/or a line) that displays a pet's average mobility. The graphical representation may include at least one mapping (e.g., data points and/or a line) that displays an average mobility of similar pets. Additionally, or alternatively, the graphical representation may also include a baseline of the average mobility of similar pets and/or a baseline of the average mobility of the pet.

The diagram 500 may include prompting the pet owner to provide one or more journal entries relating to the pet's behavior. In some embodiments, the prompting may occur in response to the pet's mobility changing. Additionally, the diagram may include displaying one or more positive alerts to provide encouragement to the user. For example, if the system determines that the pet's mobility is improving, the user interface may display a positive alert to encourage the user to continue following the pet's treatment plan.

Additionally, or alternatively, if the baseline of the pet is below the average baseline of similar pets, the system may present one or more survey questions (discussed above) to the user. If the answers to the survey questions indicate that there are no actual mobility issues, the system may then utilize the response data to improve the system. If the answers to the survey questions indicate that the pet does have mobility issues, the system may provide additional content for the user to learn more about such issues.

Additionally, or alternatively, the user interface 400 may implement an acute workflow. For example, the acute workflow may be initiated when there is a substantial (e.g., greater than 2 standard deviations) change in the pet's mobility (e.g., for more than one day). In some embodiments, the acute workflow may not be triggered when the change has occurred for more than a threshold time period. For example, if the change has occurred for 15 days, where the threshold time period is 14 days, the chronic workflow may be triggered, instead of the acute workflow. The acute workflow may include one or more alerts for display on the user interface 400. For example, the user interface 400 may display an alert when the pet's mobility drops below a particular threshold. In such a situation, the user interface 400 may display one or more prompts for the user to provide one or more journal entries. Additionally, or alternatively, the user interface 400 may display an alert when a pet's mobility improves. In such a situation, the user interface 400 may display one or more prompts for the user to provide one or more journal entries. In some embodiments, the one or more journal entries may be stored in one or more data stores for further analysis and/or review.

### Exemplary Environment

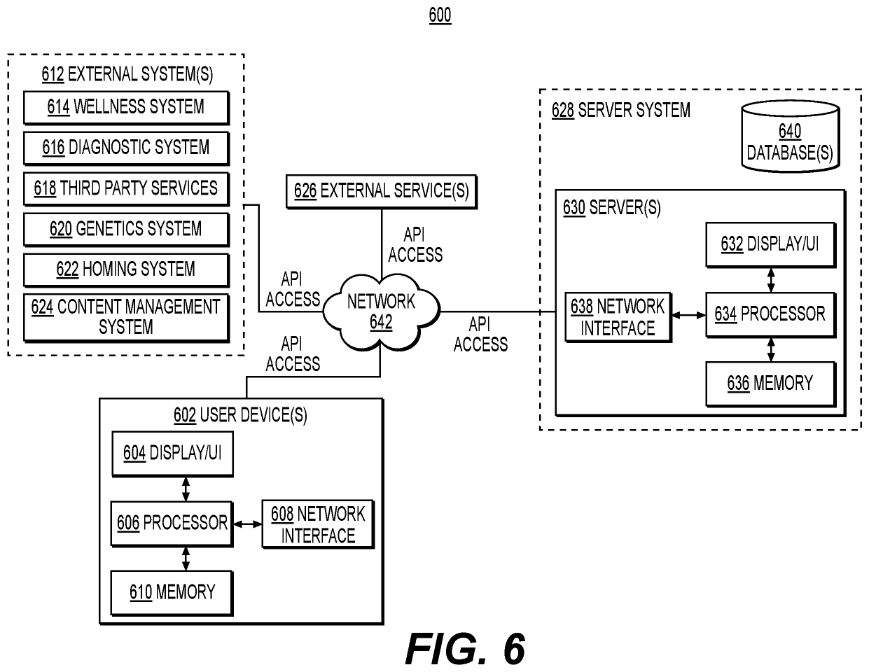

FIG. 6 depicts an exemplary environment 600 that may be utilized with the techniques presented herein. One or more user device(s) 605, one or more external system(s) 626, and one or more server system(s) 628 may communicate across a network 642. As will be discussed in further detail below, one or more server system(s) 628 may communicate with one or more of the other components of the environment 600 across network 642. The one or more user device(s) 602 may be associated with a user, e.g., a user associated with at least one pet.

In some embodiments, the components of the environment 600 are associated with a common entity, e.g., a veterinarian, clinic, animal specialist, research center, pharmaceutical company, or the like. In some embodiments, one or more of the components of the environment may be associated with a different entity than another. The systems and devices of the environment 600 may communicate in any arrangement. As will be discussed herein, systems and/or devices of the environment 600 may communicate in order to receive, send, and/or store data.

The user device 602 may be configured to enable the user to access and/or interact with other systems in the environment 600. For example, the user device 602 may be a computer system such as, for example, a desktop computer, a mobile device, a tablet, etc. In some embodiments, the user device 602 may include one or more electronic application(s), e.g., a program, plugin, browser extension, etc., installed on a memory of the user device 602. In some embodiments, the user device 602 may include a smart collar 130, a smart bed 132, a smart feeder 134, a smart litter box 136, a smart camera 138, and/or the other sensors for collecting a digital image of a pet's (e.g., canine's) life 140

The user device 602 may include a display/user interface (UI) 604, a processor 606, a memory 610, and/or a network interface 608. The user device 602 may execute, by the processor 606, an operating system (O/S) and at least one electronic application (each stored in memory 610). The electronic application may be a desktop program, a browser program, a web client, or a mobile application program (which may also be a browser program in a mobile O/S), an applicant specific program, system control software, system monitoring software, software development tools, or the like. For example, environment 600 may extend information on a web client that may be accessed through a web browser. In some embodiments, the electronic application(s) may be associated with one or more of the other components in the environment 600. The application may manage the memory 610, such as a database, to transmit streaming data to network 642. The display/UI 604 may be a touch screen or a display with other input systems (e.g., mouse, keyboard, etc.) so that the user(s) may interact with the application and/or the O/S. The network interface 608 may be a TCP/IP network interface for, e.g., Ethernet or wireless communications with the network 642. The processor 606, while executing the application, may generate data and/or receive user inputs from the display/UI 604 and/or receive/transmit messages to the server system 628, and may further perform one or more operations prior to providing an output to the network 642.

External system(s) 612 may be, for example, one or more systems that collect, manage, and/or store data corresponding to one or more pets and/or one or more pet owners. The one or more external systems may include at least one of a wellness system 614, a diagnostic system 616, a third party services system 618, a genetics system 620, a homing system 622, and/or a content management system 624. External system(s) 612 may be in communication with other device(s) or system(s) in the environment 600 over the one or more networks 642. For example, external system(s) 612 may communicate with the server system 628 via API (application programming interface) access over the one or more networks 642, and also communicate with the user device(s) 602 via web browser access over the one or more networks 642.

External service(s) 626 may be, for example, one or more third party and/or auxiliary systems that integrate and/or communicate with the server system 628 in performing various document information extraction tasks. External service(s) 626 may be in communication with other device(s) or system(s) in the environment 600 over the one or more networks 642. For example, external service(s) 626 may communicate with the server system 628 via API access over the one or more networks 642, and also communicate with the user device(s) 602 via web browser access over the one or more networks 642.

In various embodiments, the network 642 may be a wide area network (“WAN”), a local area network (“LAN”), a personal area network (“PAN”), or the like. In some embodiments, network 642 may include the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 628 may include an electronic data system, e.g., a computer-readable memory such as a hard drive, flash drive, disk, etc. In some embodiments, the server system 628 includes and/or interacts with an application programming interface for exchanging data to other systems, e.g., one or more of the other components of the environment.

The server system 628 may include a database(s) 640 and server(s) 630. The server system 628 may be a computer, system of computers (e.g., rack server(s)), and/or or a cloud service computer system. The server system may store or have access to database(s) 640 (e.g., hosted on a third party server or in memory 638). The server(s) may include a display/UI 632, a processor 634, a memory 636, and/or a network interface 638. The display/UI 632 may be a touch screen or a display with other input systems (e.g., mouse, keyboard, etc.) for an operator of the server(s) 630 to control the functions of the server(s) 630. The server system 628 may execute, by the processor 634, an operating system (O/S) and at least one instance of a servlet program (each stored in memory 636).

Although depicted as separate components in FIG. 6, it should be understood that a component or portion of a component in the environment 600 may, in some embodiments, be integrated with or incorporated into one or more other components. For example, a portion of the display 632 may be integrated into the user device 602 or the like. In some embodiments, operations or aspects of one or more of the components discussed above may be distributed amongst one or more other components. Any suitable arrangement and/or integration of the various systems and devices of the environment 600 may be used.

In general, any process or operation discussed in this disclosure that is understood to be computer-implementable, such as the processes illustrated in FIG. 3, may be performed by one or more processors of a computer system, such any of the systems or devices in the environment 600 of FIG. 6, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 6. One or more processors of a computer system may be included in a single computing device or distributed among a plurality of computing devices. A memory of the computer system may include the respective memory of each computing device of the plurality of computing devices.

### Exemplary Device

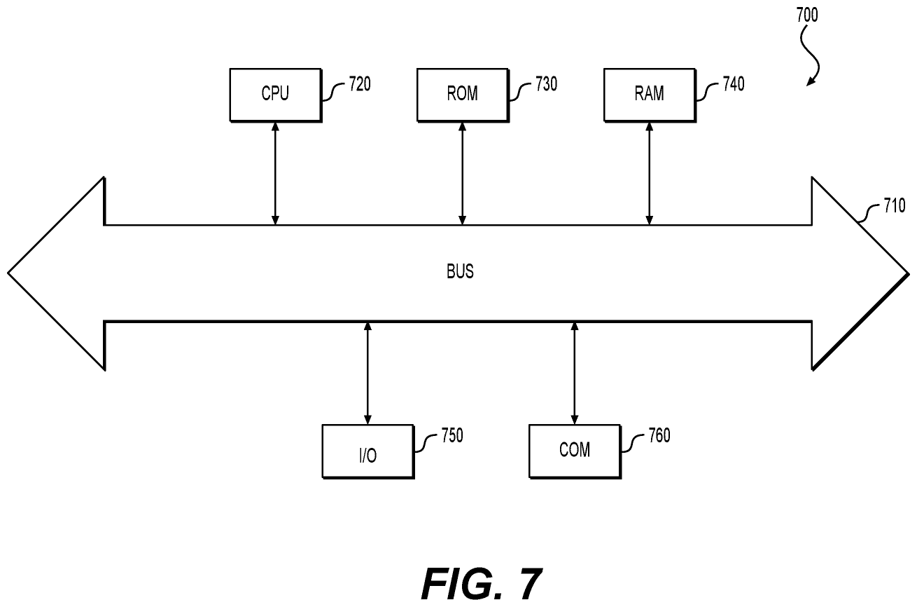

FIG. 7 is a simplified functional block diagram of a computer 700 that may be configured as a device for executing the environments and/or the methods of FIGS. 1-5, according to exemplary embodiments of the present disclosure. For example, device 700 may include a central processing unit (CPU) 720. CPU 720 may be any type of processor device including, for example, any type of special purpose or a general-purpose microprocessor device. As will be appreciated by persons skilled in the relevant art, CPU 720 also may be a single processor in a multi-core/multiprocessor system, such system operating alone, or in a cluster of computing devices operating in a cluster or server farm. CPU 720 may be connected to a data communication infrastructure 710, for example, a bus, message queue, network, or multi-core message-passing scheme.

Device 700 also may include a main memory 740, for example, random access memory (RAM), and also may include a secondary memory 730. Secondary memory 730, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 730 may include other similar means for allowing computer programs or other instructions to be loaded into device 700. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 700.

Device 700 also may include a communications interface (“COM”) 760. Communications interface 760 allows software and data to be transferred between device 700 and external devices. Communications interface 760 may include a modem, a network interface (such as an Ethernet card), a communications port, a PCMCIA slot and card, or the like. Software and data transferred via communications interface 760 may be in the form of signals, which may be electronic, electromagnetic, optical, or other signals capable of being received by communications interface 760. These signals may be provided to communications interface 760 via a communications path of device 700, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 700 also may include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

A computer system, such as a system or device implementing a process or operation in the examples above, may include one or more computing devices. One or more processors of a computer system may be included in a single computing device or distributed among a plurality of computing devices. A memory of the computer system may include the respective memory of each computing device of the plurality of computing devices.

A computer may be configured as a device for executing the exemplary embodiments of the present disclosure. For example, the computer may be configured according to exemplary embodiments of this disclosure. In various embodiments, any of the systems herein may be a computer including, for example, a data communication interface for packet data communication. The computer also may include a central processing unit (“CPU”), in the form of one or more processors, for executing program instructions. The computer may include an internal communication bus, and a storage unit (such as ROM, HDD, SDD, etc.) that may store data on a computer readable medium, although the computer may receive programming and data via network communications. The computer may also have a memory (such as RAM) storing instructions for executing techniques presented herein, although the instructions may be stored temporarily or permanently within other modules of computer (e.g., processor and/or computer readable medium). The computer also may include input and output ports and/or a display to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for canine mobility detection, the method comprising:
processing, by one or more processors, mobility data captured by a device attached to a canine, the processing including determining one or more metrics based on the mobility data, the one or more metrics including velocity information, cadence information, acceleration information, and entropy information of the canine;
analyzing, by the one or more processors, canine data corresponding to the canine to determine at least one baseline canine, wherein the at least one baseline canine is similar to the canine;
for each of the one or more metrics, comparing, by the one or more processors, the one or more metrics of the canine to one or more baseline metrics of the at least one baseline canine;
determining, by the one or more processors, one or more scores for each of the one or more metrics, the one or more scores based on a normal range of the one or more metrics from the one or more baseline metrics of the at least one baseline canine; and
displaying, by the one or more processors, at least one alert on one or more user interfaces of a user device, the at least one alert indicating that an average of the one or more scores is above a threshold.

2. The computer-implemented method of claim 1, wherein the at least one alert includes positive reinforcement.

3. The computer-implemented method of claim 1, the method further comprising:
analyzing, by the one or more processors, the mobility data to determine at least one portion of the mobility data that does not include walking data; and
removing, by the one or more processors, the at least one portion from the mobility data.

4. The computer-implemented method of claim 1, the analyzing including:
segmenting, by the one or more processors, the mobility data into a plurality of windows based on at least one time interval;
analyzing, by the one or more processors, each of the plurality of windows to determine whether a threshold number of the plurality of windows is below a window threshold; and
removing, by the one or more processors, each of the plurality of windows that falls below the threshold.

5. The computer-implemented method of claim 1, wherein the normal range includes an upper mobility bound and a lower mobility bound.

6. The computer-implemented method of claim 5, the analyzing further including:
determining, by the one or more processors, a highest oscillation frequency of the plurality of windows;
analyzing, by the one or more processors, the highest oscillation frequency to determine whether the highest oscillation frequency falls outside of an oscillation range; and
in response to determining that the highest oscillation frequency does fall outside of the oscillation range, removing, by the one or more processors, the window of the plurality of windows that corresponds to the highest oscillation frequency from the mobility data.

7. The computer-implemented method of claim 1, wherein the device is attached to a collar of the canine.

8. The computer-implemented method of claim 1, the method further comprising:
receiving, by the one or more processors, canine veterinary data associated with the canine from one or more external systems;
receiving, by the one or more processors, baseline canine veterinary data associated with the at least one baseline canine from one or more data stores;
analyzing, by the one or more processors, the canine veterinary data, the one or more scores, the baseline canine veterinary data, and the one or more baseline metrics;
based on the analyzing, determining, by the one or more processors, that the canine has a mobility issue; and
displaying, by the one or more processors, at least one mobility alert indicating the mobility issue on the one or more user interfaces of the user device.

9. The computer-implemented method of claim 8, the canine veterinary data including canine medication data including at least one medication dosage amount, at least one medication description, at least one medication administrator, or at least one medication administration timestamp.

10. The computer-implemented method of claim 1, the canine data including age data of the canine, breed data of the canine, weight data of the canine, one or more risk factors of the canine, or medical history of the canine.

11. A computer system for canine mobility detection, the computer system comprising:
at least one memory storing instructions; and
at least one processor configured to execute the instructions to perform operations comprising:
processing mobility data captured by a device attached to a canine, the processing including determining one or more metrics based on the mobility data, the one or more metrics including velocity information, cadence information, acceleration information, and entropy information of the canine;
analyzing canine data corresponding to the canine to determine at least one baseline canine, wherein the at least one baseline canine is similar to the canine;
for each of the one or more metrics, comparing the one or more metrics of the canine to one or more baseline metrics of the at least one baseline canine;
determining one or more scores for each of the one or more metrics, the one or more scores based on a normal range of the one or more metrics from the one or more baseline metrics of the at least one baseline canine; and
displaying at least one alert on one or more user interfaces of a user device, the at least one alert indicating that an average of the one or more scores is above a threshold.

12. The computer system of claim 11, wherein the at least one alert includes positive reinforcement.

13. The computer system of claim 11, the operations further comprising:
analyzing the mobility data to determine at least one portion of the mobility data that does not include walking data; and
removing the at least one portion from the mobility data.

14. The computer system of claim 11, the analyzing including:
segmenting the mobility data into a plurality of windows based on at least one time interval;
analyzing each of the plurality of windows to determine whether a threshold number of the plurality of windows is below a window threshold; and
removing each of the plurality of windows that falls below the threshold.

15. The computer system of claim 11, wherein the normal range includes an upper mobility bound and a lower mobility bound.

16. The computer system of claim 15, the analyzing further including:
determining a highest oscillation frequency of the plurality of windows;
analyzing the highest oscillation frequency to determine whether the highest oscillation frequency falls outside of an oscillation range; and
in response to determining that the highest oscillation frequency does fall outside of the oscillation range, removing the window of the plurality of windows that corresponds to the highest oscillation frequency from the mobility data.

17. The computer system of claim 11, wherein the device is attached to a collar of the canine.

18. A non-transitory computer-readable medium storing instructions that, when executed by at least one processor, cause the at least one processor to perform operations for canine mobility detection, the operations comprising:
processing mobility data captured by a device attached to a canine, the processing including determining one or more metrics based on the mobility data, the one or more metrics including velocity information, cadence information, acceleration information, and entropy information of the canine;
analyzing canine data corresponding to the canine to determine at least one baseline canine, wherein the at least one baseline canine is similar to the canine;
for each of the one or more metrics, comparing the one or more metrics of the canine to one or more baseline metrics of the at least one baseline canine;
determining one or more scores for each of the one or more metrics, the one or more scores based on a normal range of the one or more metrics from the one or more baseline metrics of the at least one baseline canine; and
displaying at least one alert on one or more user interfaces of a user device, the at least one alert indicating that an average of the one or more scores is above a threshold.

19. The non-transitory computer-readable medium of claim 18, wherein the device is attached to a collar of the canine.

20. The non-transitory computer-readable medium of claim 18, the canine data including age data of the canine, breed data of the canine, weight data of the canine, one or more risk factors of the canine, or medical history of the canine.
